(19) United States
(12) Patent Application Publication
(10) Pub. No.: US 2018/0311463 A1
(43) Pub. Date: Nov. 1, 2018

(54) SENSORY COASTER CHAIR FOR APPLYING DEEP TOUCH PRESSURE AND METHOD OF USE

(71) Applicant: The Sensory Chair Company, Inc., Overland Park, KS (US)

(72) Inventors: Stuart T. Jackson, Overland Park, KS (US); Keith E. Manbeck, Gardner, KS (US); Noah W. Scheier, Overland Park, KS (US); Evan W. Whited, Overland Park, KS (US)

(21) Appl. No.: 15/965,030

(22) Filed: Apr. 27, 2018

Related U.S. Application Data
(60) Provisional application No. 62/490,810, filed on Apr. 27, 2017.

(51) Int. Cl. A61M 21/02; A47C 1/024; A61H 15/00

(52) U.S. Cl. CPC A61M 21/02; A47C 1/024; A61M 2021/0022; A61H 2203/0437; A61H 15/00

(57) ABSTRACT

A sensory chair and method of use. The system generally includes a chair portion and a roller portion. The roller portion is connected to the chair portion by a pair of pivotable arms located on either side of the chair portion and the roller portion. The roller could be of any usable diameter. The seat portion could be upright, reclined, or any variation therebetween.

### CROSS-REFERENCE TO RELATED APPLICATION

This application claims priority in U.S. Provisional Patent Application No. 62/490,810 Filed Apr. 27, 2017, which is incorporated herein by reference.

### BACKGROUND OF THE INVENTION

### 1. Field of the Invention

The present invention relates generally to sensory furniture, and more specifically to a sensory chair that applies deep touch pressure to a person's body, and the method to manufacture and use the same. Such furniture can be used in, but is not limited to, therapies related to neurodevelopmental disorders such as sensory processing disorder, autism, and other neurological, neuropsychological and physical conditions.

### 2. Description of the Related Art

Deep touch pressure is a form of tactile sensory input, often provided by firm holding, firm stroking, hugging, swaddling, and squeezing. A non-invasive and easily applied therapeutic method, deep touch pressure generally provides a calming effect for the recipient. A growing body of literature shows that deep touch pressure is helpful for managing anxiety and reducing maladaptive behaviors in people with cognitive developmental disorders, sensory processing disorder, psychological disorders, and certain other neurological and physical conditions. Research also shows that deep touch pressure can improve the quality of life for people experiencing anxiety, pain and unrest

Several devices have been designed and developed to provide continuous deep touch pressure. Such devices include weighted blankets, therapy vests and other sensory based interventions which provide relatively low amounts of pressure. In the past, deep touch pressure machines have been developed to provide pressure to persons exhibiting oversensitivity to human touch. One such machine developed by Krauss (1987) was designed as having two air mattresses surrounded by a canvas wrap which was connected to a pulley. A person would lie between the two mattresses and pull on the rope connected to the pulley to increase the pressure exerted on them. Krauss, Am. J. Occup. Ther., The Effects of Deep Pressure on Anxiety, 41:366-373 (1987). Another such machine that has been widely used is the squeeze machine developed by Temple Grandin. The squeeze machine provided increased pressure compared to the Krauss device. The squeeze machine included two padded side boards hinged at the bottom to form a “V” shape, similar to a cattle squeeze chute. A lever-operated pneumatic valve is connected to an air cylinder that pulls the boards together. The lever allows the user to regulate the amount of pressure applied. Grandin, J. Child Adolescent Psychopharmacology, Calming Effects of Deep Touch Pressure in Patients with Autistic Disorder, College Students, and Animals, 2(1): 63-72 (1992). While these devices accomplish the task of providing deep touch pressure, there remained a need for a practical, lightweight, quiet device that provided variable deep touch pressure consistent with the needs of a user.

A sensory treatment device system known as the Sensory Lounger (USPTO Publication No. US-2016-0158089), was developed to provide deep touch pressure to a person in a comfortable manner, adaptable to persons of varying shapes and sizes, in which the person lay down between two air tubes which were subsequently inflated to provide deep touch pressure.

A sensory treatment device system known as the Sensory Upright (USPTO Publication No. US-2016-0158090), was developed to provide deep touch pressure to a person in a comfortable manner, adaptable to persons of varying shapes and sizes, in which the person sat upright between two air tubes which were subsequently inflated to provide deep touch pressure.

While the Sensory Lounger and Sensory Upright system provided deep touch pressure to a person employing compressive deep touch pressure provided by two inflatable air tubes, the present invention allows a person to sit up in an upright or reclined position while receiving pressure applied via a roller. Heretofore there has not been available a system or method for a sensory treatment device with the advantages and features of the present invention.

### BRIEF SUMMARY OF THE INVENTION

The present invention generally provides a sensory chair and method of use. The system generally includes a chair portion and a roller portion. The roller portion is connected to the chair portion by a pair of pivotable arms located on either side of the chair portion and the roller portion.

The pivotable arms connecting the roller portion to the chair portion can connect to the chair portion at different positions. In one embodiment, the pivotable arms connect to the chair portion at a single point on either side of the chair, just below where the seat meets the back of the chair. This allows the roller portion to be extended in front of the chair portion, allowing a person (the “user”) to enter the device by stepping between the front of the chair and the roller portion and sitting down. In this embodiment, the handles of the roller portion can then be used by a second person (the “operator”) to lift up the roller portion and place it on the user's lap.

In another embodiment, the pivotable arms connect to the chair portion at a single point on either side of the chair, at the top of the back of the chair portion. This allows the roller portion to be extended behind the chair portion, allowing the user to enter the device by simply sitting down in the chair. In this embodiment, the roller portion is then lifted up and over the back of the chair portion by the operator and placed in front of the user's body and onto their lap.

Other embodiments have the pivotable arms connected to the chair portion at either of the previous two positions or at other positions on the chair portion.

When the user is seated, the operator applies deep touch pressure to the user by using the handles of the roller portion to push the roller against the user's body. The roller can be pushed up and down the users body or in a side to side motion. Stretchable bands can also be attached between the chair and the roller portion to pull the roller toward the user for additional pressure.

To prevent the chair section from flipping, rubber stoppers are placed on the back of the chair portion and makes it easier for a user to lift and move the roller out of the way to enter or exit the chair.

When the deep touch pressure session is finished, the operator places the roller section either in front of the chair section or behind the chair section and the o is then free to exit the device.

### BRIEF DESCRIPTION OF THE DRAWINGS

The drawings constitute a part of this specification and include exemplary embodiments of the present invention illustrating various objects and features thereof.

FIG. 1 is a back-left isometric view of a preferred embodiment of the present invention.

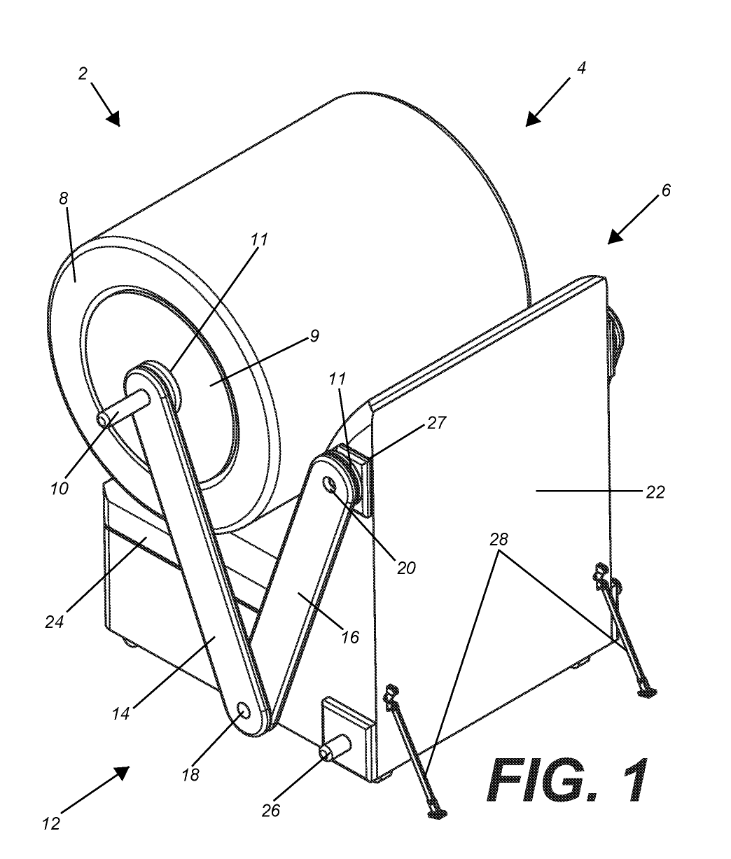

FIG. 2 is a back-right isometric view thereof.

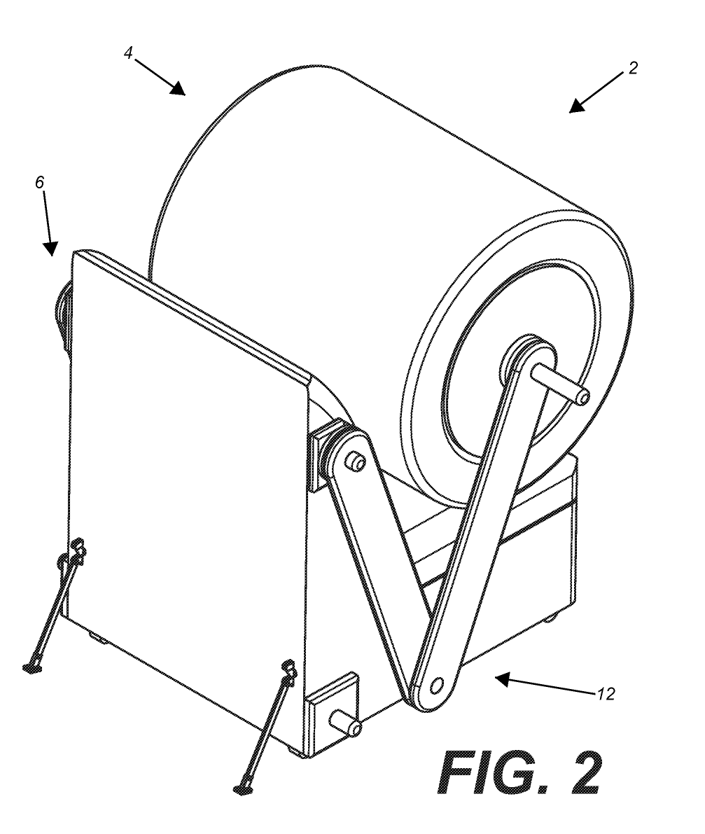

FIG. 3 is a front-left isometric view thereof.

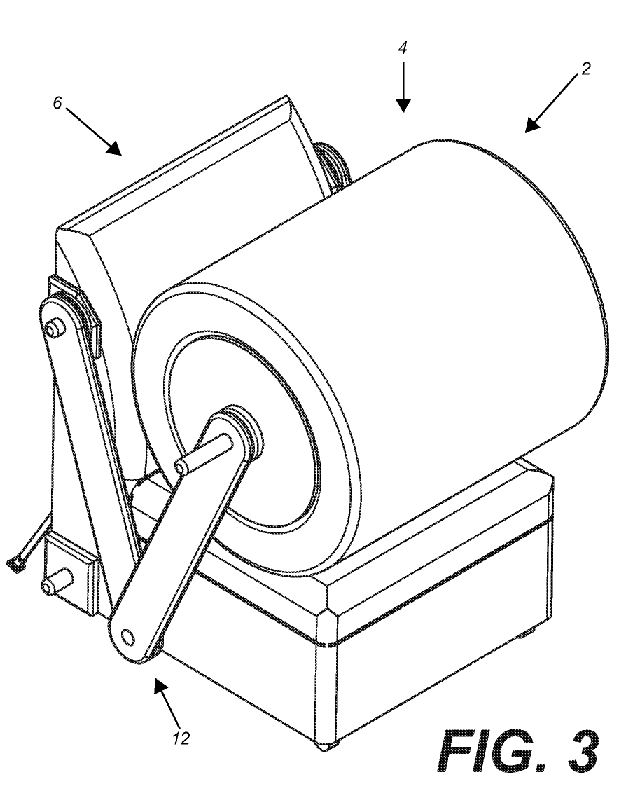

FIG. 4 is a front-right isometric view thereof.

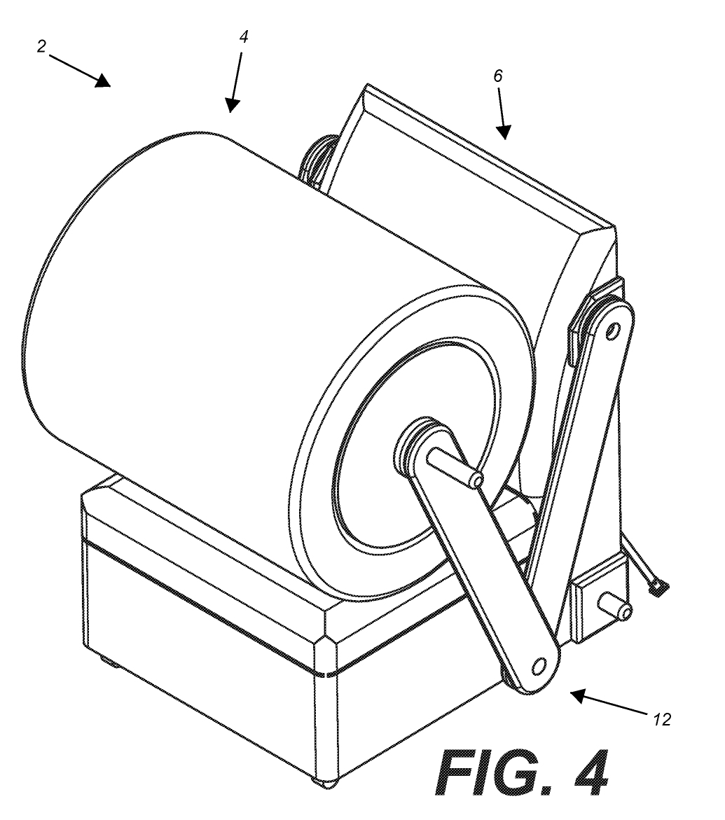

FIG. 5 is a back-right exploded isometric view thereof.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Sensory Coaster Chair 2

As shown in the figures, the present invention is a sensory coaster chair 2 which includes a chair portion 6, a roller portion 4, and a pair of pivotable arms 12 which connects the chair portion and the roller portion. The roller portion 4 is capable of being lifted above a user's head while the user is sitting in the chair portion 6, or in front of the chair section, such that the user may enter or exit the chair.

The roller 8 applies pressure to the user when the user is seated in the chair. Each end of the roller may include handles 10 which allow the operator to move the roller over the user's body, thereby applying pressure. Stretchable bands (not shown) may be used to provide additional pressure. These bands could stretch before the ends of the handles 10 to a corresponding pair of pegs 26 located on the rear of the chair portion 6.

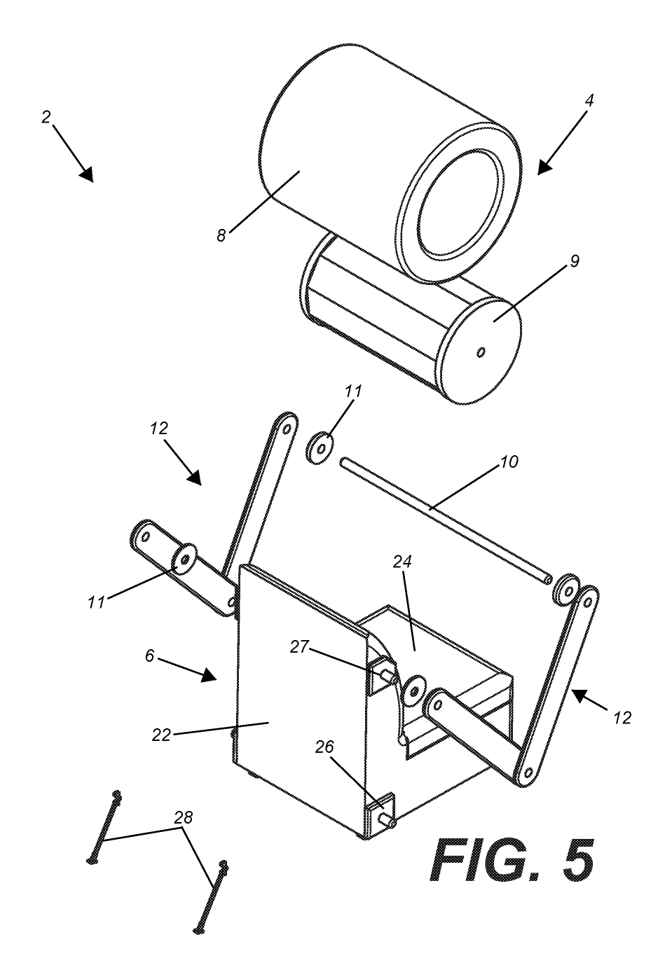

The coaster portion 4 has an internal drum 9 which is inserted into the roller 8 to help maintain the shape of the roller. The handle 10, as shown in FIG. 5, may be a dowel which passes completely through the drum 9 to provide the handles on either end of the coaster portion 4. A washer element 11 helps to allow the drum 9 to rotate against the pivotable arms 12 located on either side of the coaster. The roller 8 is made of a firm yet padded material, such as foam, cloth, or other suitable material. It is designed to provide soft but firm pressure across large portions of the user's body.

The seat portion 6 has a back portion 22, a seat portion 24, and optional kick stands 28 to help secure the seat portion when the coaster is in use. The back portion 22 could be upright as shown, or it could be in any various state of recline. An embodiment of the present invention could have a freely reclining back seat portion. The seat portion may include padding for comfort, as may the back portion.

The pivotable arms 12 include a first arm 14 and a second arm 16 joined at a pivot point 18. The second arm 16 also joins at a peg 27 located on the seat portion 6 and forms a second pivot point 20. A washer 11 is inserted between the arm 16 and the peg 27.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A sensory coaster chair system comprising:
a chair portion having a seat and a back;
a coaster portion having a cylindrically shaped roller having a first end and a second end, a first handle located in proximity of said first end of said roller, and a second handle located in proximity of said second end of said roller;
a pair of pivotable arms connecting each of said first and second ends of said coaster to a respective side of said chair portion; and
said coaster configured to be rolled against a body placed within said chair portion, said coaster further configured to apply deep touch pressure to at least one portion of said body.

2. The system of claim 1, further comprising:
said coaster portion comprising an external roller and an internal cylindrical drum; and
a washer placed adjacent to each of said first and second ends of said coaster between said cylindrical drum and a respective one of said pair of pivotable arms.

3. The system of claim 1, further comprising:
each one of said pair of pivotable arms comprising a first arm member and a second arm member joined at a pivot point;
wherein each respective first arm member is connected to a respective one of said first and second ends of said coaster portion; and
wherein each respective second arm member is connected to said chair portion.

4. The system of claim 1, further comprising:
said chair portion back being in a fully reclined orientation, wherein said back is parallel to said chair portion.

5. The system of claim 1, further comprising:
said chair portion being in a fully upright orientation, wherein said back is perpendicular to said seat portion.

6. The system of claim 1, further comprising:
said back of said chair portion is placed in an angle between ninety degrees and one hundred eighty degrees in relation to said seat.

7. The system of claim 6, wherein said back is freely pivotable about said chair portion between ninety degrees and one hundred eighty degrees in relation to said seat.

8. The system of claim 1 further comprising a pair of bands, each band connecting a respective one of said first and second sides of said coaster to said chair portion.

9. A method of operating a sensory coaster, the method comprising the steps:
connecting a coaster portion to a chair portion via a pair of pivotable arms;
placing a body within said chair portion, said chair portion having a seat and a back;
moving said coaster portion over said chair portion and placing said coaster portion against a portion of said body; and
delivering deep touch pressure to said portion of said body.

10. The method of claim 9, further comprising the step of rolling said coaster portion about said body.

11. The method of claim 9, further comprising the step of connecting a pair of bands between said coaster portion and said chair portion, said pair of bands thereby pulling said coaster portion against said body.

12. The method of claim 9, wherein:
said coaster portion comprising an external roller and an internal cylindrical drum; and
a washer placed adjacent to each of said first and second ends of said coaster between said cylindrical drum and a respective one of said pair of pivotable arms.

13. The method of claim 9, wherein:
each one of said pair of pivotable arms comprising a first arm member and a second arm member joined at a pivot point;
wherein each respective first arm member is connected to a respective one of said first and second ends of said coaster portion; and
wherein each respective second arm member is connected to said chair portion.

14. The method of claim 9, wherein said chair portion back being in a fully reclined orientation, wherein said back is parallel to said chair portion.

15. The method of claim 9, wherein said chair portion being in a fully upright orientation, wherein said back is perpendicular to said seat portion.

16. The method of claim 9, wherein said back of said chair portion is placed in an angle between ninety degrees and one hundred eighty degrees in relation to said seat.

17. The method of claim 9, further comprising the step of pivoting said back portion about said chair portion such that said back portion is tilted to an angle between ninety degrees and one hundred eighty degrees in relation to said seat.
